(19) United States
(12) Patent Application Publication
(10) Pub. No.: US 2010/0198289 A1
(43) Pub. Date: Aug. 5, 2010

(54) Method and device for the prevention of sudden unexpected death in epilepsy (SUDEP)

(75) Inventors: Nader Kameli, Hugo, MN (US); Willard Wilson, Quebec (CA)

Correspondence Address: POLSINELLI SHUGHART PC, 700 W. 47TH STREET, SUITE 1000, KANSAS CITY, MO, 64112-1802, US

(73) Assignee: Victhom Human Bionics, Inc., Saint-Augustin-de-Desmaures (CA)

(21) Appl. No.: 12/364,363

(22) Filed: Feb. 2, 2009

(51) Int. Cl. A61N 1/362; A61B 5/00; A61B 5/0476; A61N 1/36; A61M 5/00

(52) U.S. Cl. 607/14; 600/301; 600/544; 607/42; 604/890.1

(57) ABSTRACT

A method and system for circumventing sudden unexpected death in epilepsy (SUDEP) by monitoring a plurality of indicators related to the likelihood of SUDEP, processing and monitoring these indicators for conditions predisposing to SUDEP, selecting a preconfigured treatment to treat the existing set of high-risk conditions, and delivering or triggering apnea and/or seizure treatment of a preventive or therapeutic nature to prevent SUDEP.

### TECHNICAL FIELD

The present invention relates to methods and systems for the prevention and treatment of sudden unexpected death in epilepsy.

### BACKGROUND

Epilepsy is the most common neurological disorder, affecting about 1% of the population worldwide. The death rate among epileptics is about 3 times that of age-matched cohorts. This statistic is partly explained by consequences of epilepsy, but in a surprising number of cases, there is no obvious cause of death. Sudden death from unknown causes occurs in epileptics at a rate 24 times that of the general population. In patients with severe, refractory epilepsy, sudden unexpected death in epilepsy (SUDEP) may account for 50% of all deaths.

While there is no toxicological or anatomical explanation for SUDEP, evidence suggests respiratory failure during seizure. Eyewitness reports indicate seizure and apnea shortly before death. Postmortem evidence supports this conclusion in the majority of SUDEP cases.

Postmortem evidence also implicates sleep in a disproportionate number of cases, and a relationship between sleep and seizure has long been known. Seizure activity increases during sleep and the synchronized cortical activity of non-rapid eye movement (NREM) sleep is thought to promote seizure initiation. Conversely, sleep deprivation is correlated with increased seizure activity and treatment of sleep-related breathing disorders (SRBDs) in epileptics is associated with improved seizure control.

Polysomnography of sleeping epileptics indicates that significant respiratory difficulty during seizures is common. Central apnea (CA), a decreased central respiratory drive to the diaphragm is most common, but obstructive apnea (OA), a physical blockage of the upper airway, is also observed.

Fatal apnea has been observed during experimentally induced seizures in animal models. Tracheostomy does not prevent death during induced seizures in sheep, suggesting that the fatal apnea is central in nature. However, nerve recordings in rat and piglet models suggest that both obstructive and central mechanisms play a role in fatal ictal apnea. Death in animal models of epilepsy can be prevented by pulmonary resuscitation or by placing animals in an oxygenated environment during seizure.

Evidence suggests that SUDEP in humans may also be preventable by intervention. For example, the presence of a caregiver during sleep significantly decreases the incidence of SUDEP in high-risk patients. Intervention might be as simple as waking the patient or helping them to change position. Discouraging prone sleeping has cut the incidence of sudden infant death syndrome (SIDS) by about 50%. Similar intervention may also be effective for SUDEP, where postmortem evidence indicates prone sleeping in the majority of cases.

Medical devices that detect apnea by monitoring bioelectric activity of the certain breathing muscles, or their efferent nerves have been described, as have devices that detect apnea by monitoring implanted sensors for indications of, for example, thoracic pressure or blood oxygenation.

Medical devices that treat apnea using drug delivery, atrial overdrive pacing or electrical stimulation of the nerves or muscles that control respiratory activities have been described. Electrical stimulation has been used to maintain upper airway patency by activating airway muscles or the efferent nerves controlling them. Electrical stimulation of the diaphragm, intercostal muscles, or their efferent nerves has also been described.

Medical devices have been developed to prevent epilepsy using electrical stimulation of the cortex, deep brain structures, or the vagus nerve. Most operate in a continuous treatment mode, thought to decrease the probability of seizure through long-term effects on the nervous system. Treatment parameters are initially calibrated by a physician programmer and remain fixed over normal operation. For example, deep brain stimulators deliver epilepsy treatment continuously at a pulse rate of about 130 Hz. Vagus nerve stimulators (VNS) deliver bursts of pulses about 30 seconds long, at intervals of about 5 minutes. Also described is timing VNS stimulation to occur during, or immediately before a seizure to decrease the duration and severity of the seizure. In these cases, stimulation is manually triggered by the patient or a caregiver in response to an aura or an observed seizure. Automated seizure prediction and detection algorithms have been developed to similarly time seizure treatment to precede or coincide with seizures.

However, a need remains for methods and systems to address the threat of SUDEP in patients at risk of experiencing both seizure and apnea.

### SUMMARY

Given the close relationship between apnea, seizure, and SUDEP, the present invention prevents SUDEP by preventing and/or treating both apnea and seizure, ensuring normal recovery from either or both events. The invention is based in part on the realization that monitoring of multiple physiological indicators predisposing to SUDEP, particularly but not limited to apnea and seizure activity, can be used to detect the need for and then apply treatment for SUDEP. The detection is performed by processing inputs derived from the multiple physiological indicators of SUDEP to determine SUDEP likelihood, and generating an output instructing a SUDEP treatment when SUDEP likelihood is sufficiently high. The treatment may precede or coincide with seizure and/or apnea in order to prevent SUDEP. The treatment can include treatment for apnea, or treatment for seizure or both depending on the subject's particular state, patient history and treatment calibration as may be determined by a physician or other medical caregiver.

According to an illustrative embodiment of the present invention, there is provided a method to prevent SUDEP in a subject in need thereof comprising acquiring from the subject an electrical signal representing each of at least two SUDEP indicators, using the electrical signals to compute a SUDEP index, and when the SUDEP index meets a predetermined value, generating an electrical signal representing occurrence of a SUDEP-related event.

According to another illustrative embodiment of the present invention, there is provided a method to prevent SUDEP in a subject in need thereof, the method comprising acquiring from the subject an electrical signal representing each of at least two SUDEP indicators, using the electrical signals to compute a SUDEP index, determining when one of the at least two SUDEP indicators is an apnea indicator and generating an electrical signal representing presence or absence of apnea in the subject, determining when one of the at least two SUDEP indicators is a seizure indicator and generating an electrical signal representing presence or absence of seizure in the subject, when the SUDEP index meets a predetermined value, generating a signal representing occurrence of a SUDEP-related event, and when apnea is present and seizure is absent, generating an electrical signal instructing delivery of a SUDEP treatment comprising therapeutic treatment for apnea and preventative treatment for seizure.

According to another illustrative embodiment of the present invention, there is provided a method to prevent SUDEP when seizure has been detected by delivering or triggering preventative treatment for apnea and therapeutic treatment for seizure.

According to another illustrative embodiment of the present invention, there is provided a method to prevent SUDEP in a subject in need thereof, the method comprising acquiring from the subject an electrical signal representing each of at least two SUDEP indicators, using the electrical signals to compute a SUDEP index, determining when one of the at least two SUDEP indicators is a seizure indicator and generating an electrical signal representing presence or absence of seizure in the subject, determining when one of the at least two SUDEP indicators is an apnea indicator and generating an electrical signal representing presence or absence of apnea in the subject, when the SUDEP index meets a predetermined value, generating a signal representing occurrence of a SUDEP-related event, when apnea is absent and seizure is present, generating an electrical signal instructing delivery of a SUDEP treatment comprising preventative treatment for apnea and therapeutic treatment for seizure.

According to another illustrative embodiment of the present invention, there is provided method to prevent SUDEP in a subject in need thereof, the method comprising acquiring from the subject an electrical signal representing each of at least two SUDEP indicators, using the electrical signals to compute a SUDEP index, determining when one of the at least two SUDEP indicators is a seizure indicator and generating an electrical signal representing presence or absence of seizure in the subject, determining that one of the at least two SUDEP indicators is an apnea indicator and generating an electrical signal representing presence or absence of apnea in the subject, when the SUDEP index meets a predetermined value, generating a signal representing occurrence of a SUDEP-related event, when apnea is present and seizure is present, generating an electrical signal instructing delivery of a SUDEP treatment comprising therapeutic treatment for apnea and therapeutic treatment for seizure.

According to another illustrative embodiment of the present invention, there is provided a method to prevent respiratory failure in a subject suffering from a seizure, the method comprising acquiring from the subject an electrical signal representing each of at least two SUDEP indicators wherein at least one SUDEP indicator is a seizure indicator, using the electrical signals to compute a SUDEP index, and when the SUDEP index meets a predetermined value, generating an electrical signal representing occurrence of a SUDEP-related event.

According to another illustrative embodiment of the present invention, there is provided a method to prevent SUDEP in a subject in need thereof, the method comprising, wherein the subject is treated with vagal nerve stimulation (VNS), preventing obstructive apnea in the subject by acquiring from the subject an electrical signal representing each of at least two SUDEP indicators wherein at least one of the SUDEP indicators indicates respiratory state, using the electrical signals to compute a SUDEP index, when the SUDEP index meets a predetermined value, generating an electrical signal representing a command to periodically deliver the VNS to the subject in synchrony with the expiration of the subject.

According to other illustrative embodiments of the present invention, also provided are systems for implementing the above-described methods.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE DRAWINGS

Non-limitative illustrative embodiments of the invention will now be described by way of example only with reference to the accompanying drawings, in which:

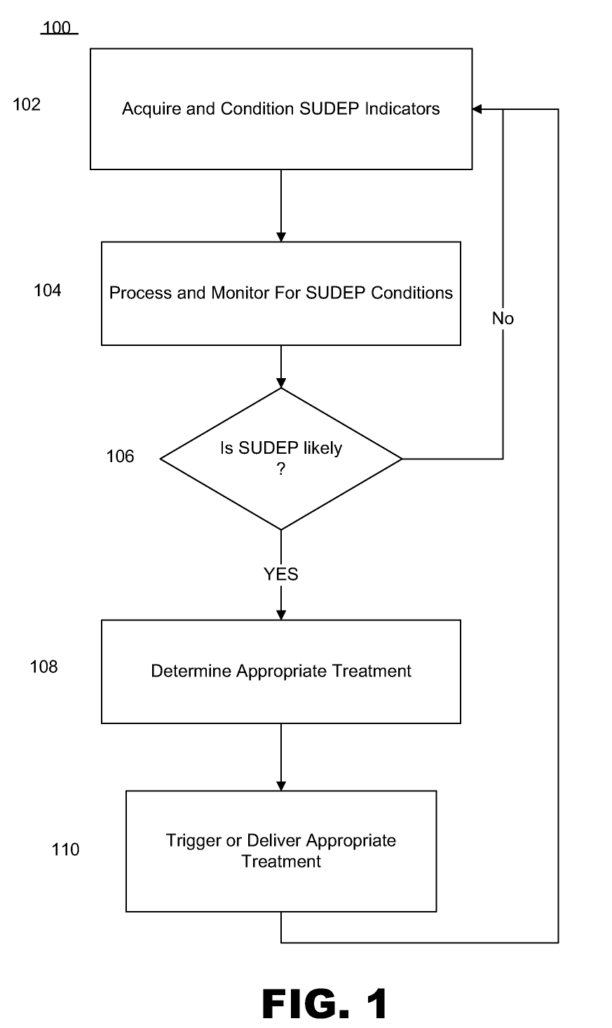

FIG. 1 is a flow chart of a method to prevent sudden death in epileptic patients;

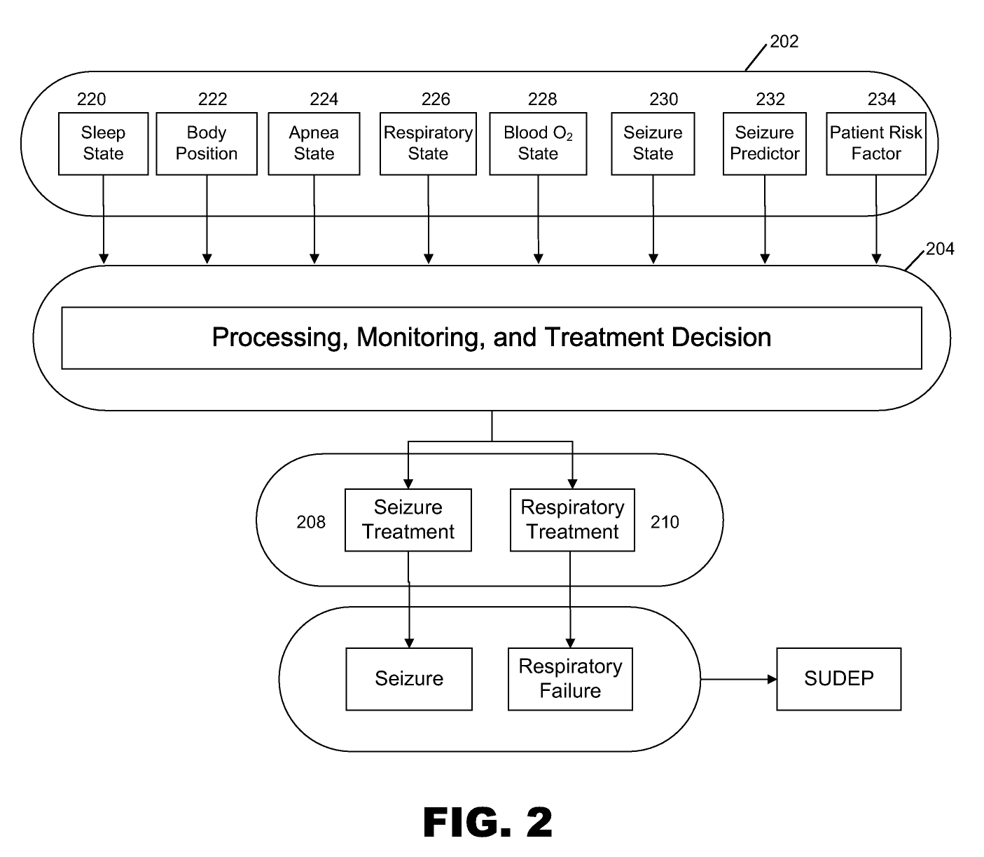

FIG. 2 is a schematic representation of a method to prevent and/or treat seizure and apnea leading to sudden death in epileptic patients;

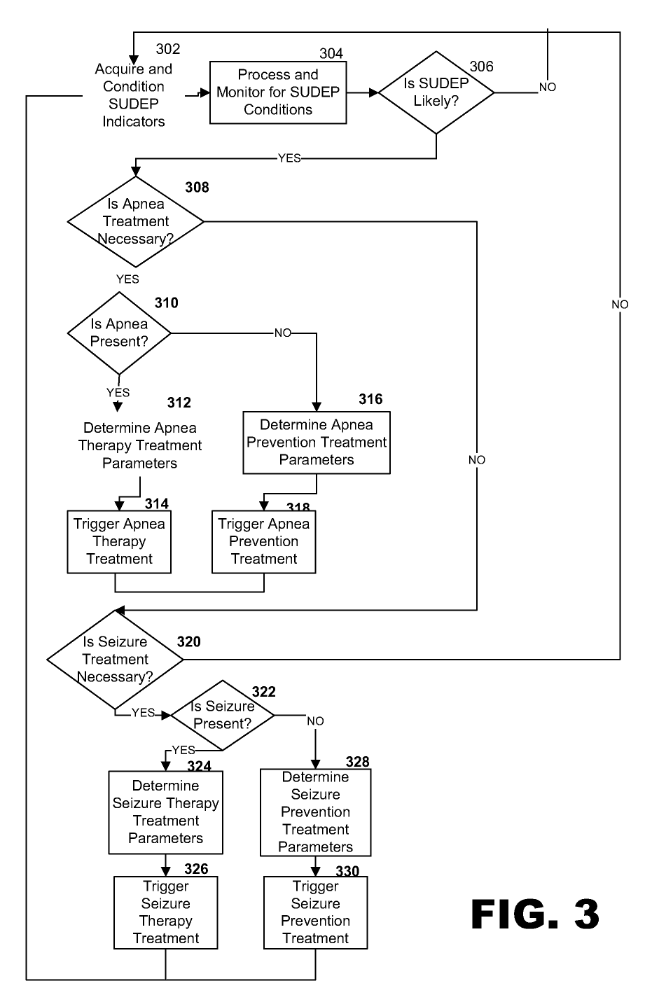

FIG. 3 is a flow chart of a method to determine appropriate seizure and/or apnea treatment to prevent sudden death in epileptic patients;

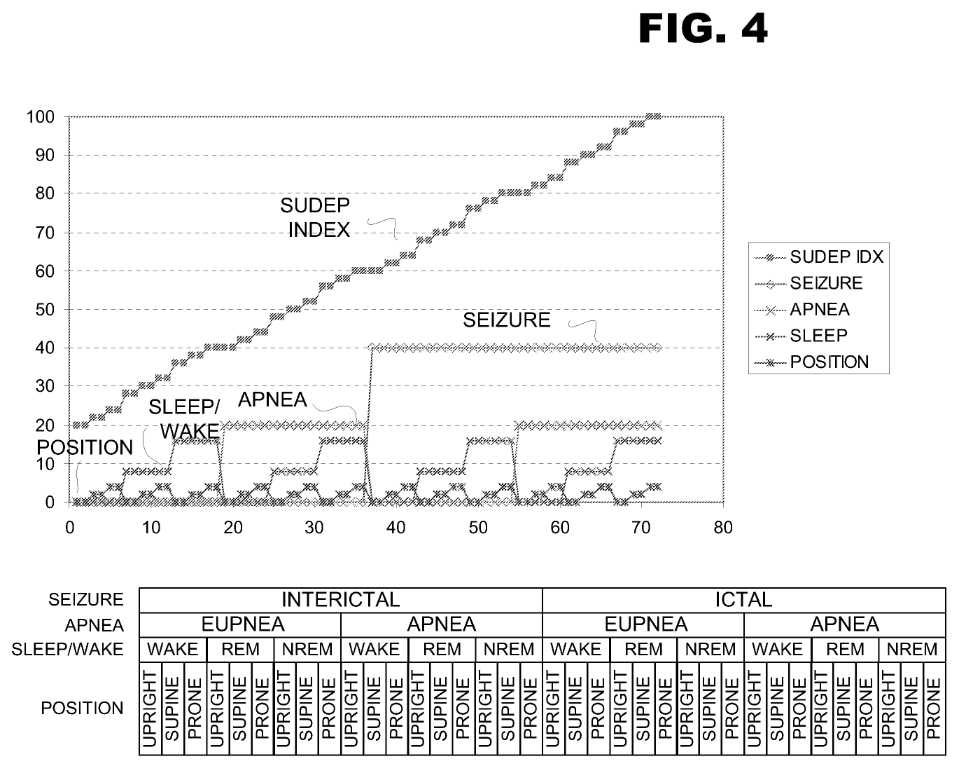

FIG. 4 is a graph illustrating a method to calculate an index of SUDEP likelihood using a weighted linear combination of indicator states;

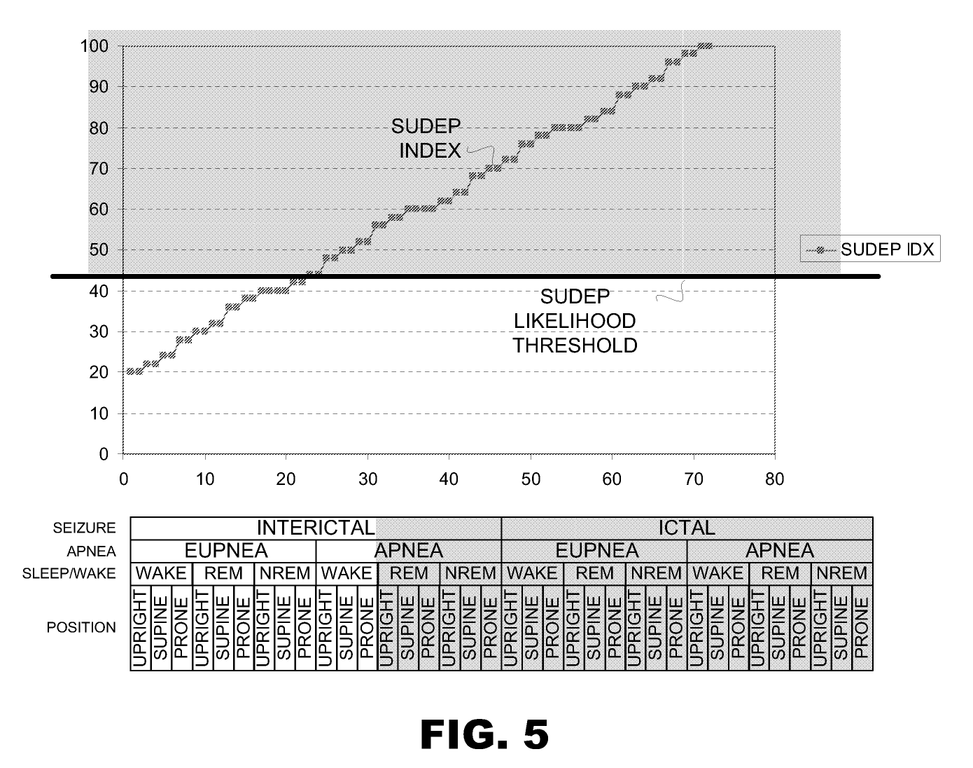

FIG. 5 is a graph illustrating a method to determine when SUDEP is likely using a thresholding algorithm and the index of SUDEP likelihood;

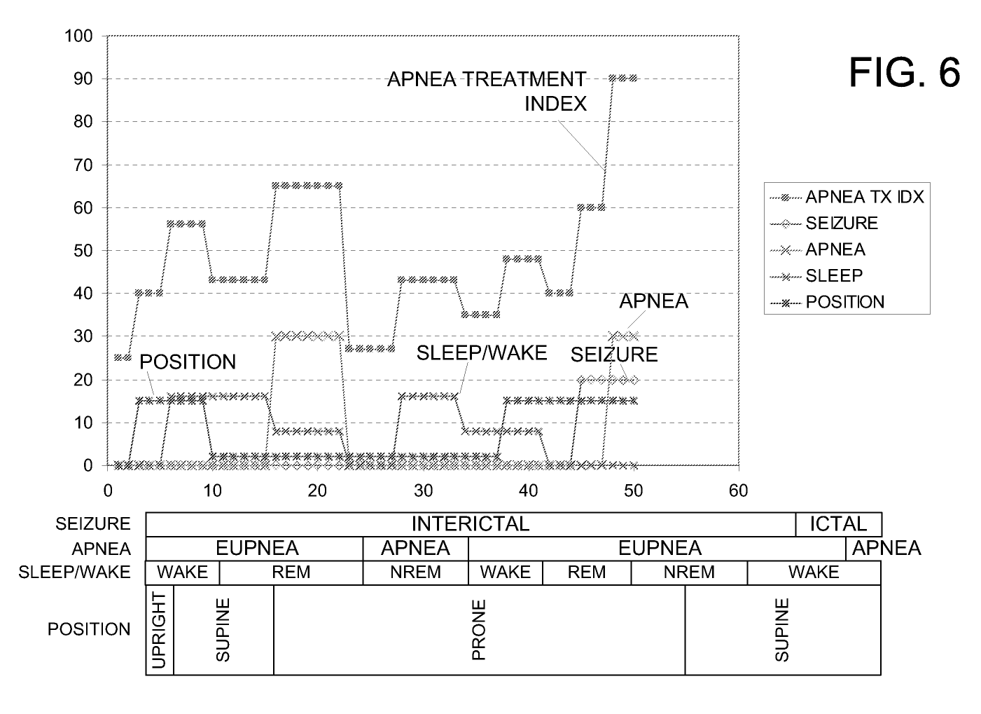

FIG. 6 is a graph illustrating a method to calculate an apnea treatment index using a weighted linear combination of indicator states;

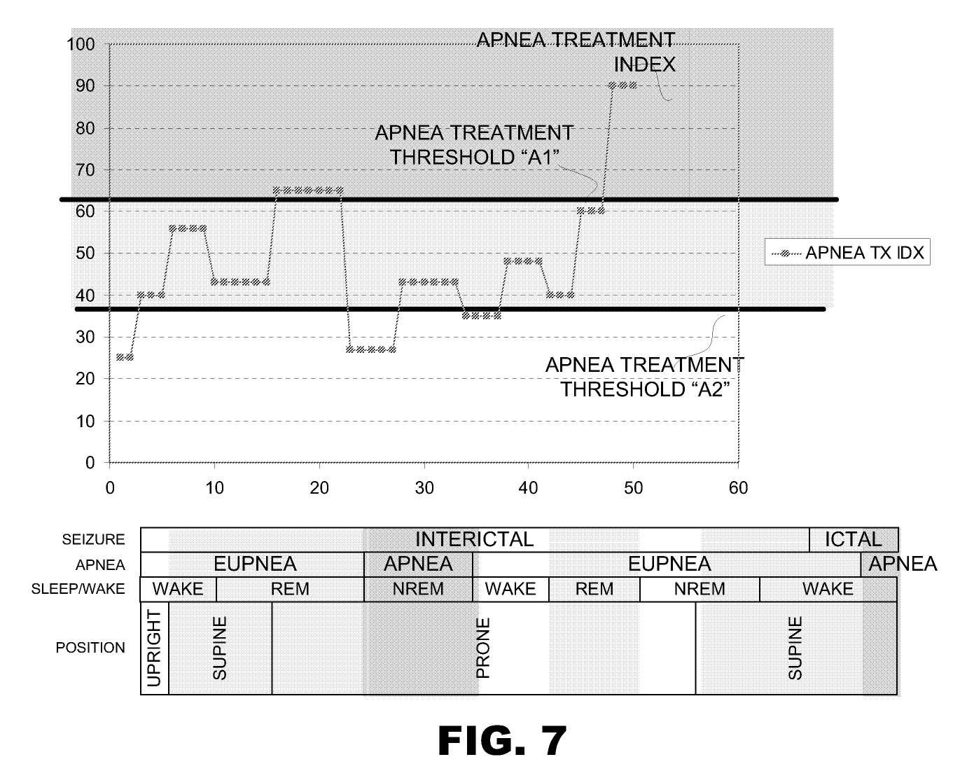

FIG. 7 is a graph illustrating a method to determine appropriate apnea treatments using a thresholding algorithm and the apnea treatment index;

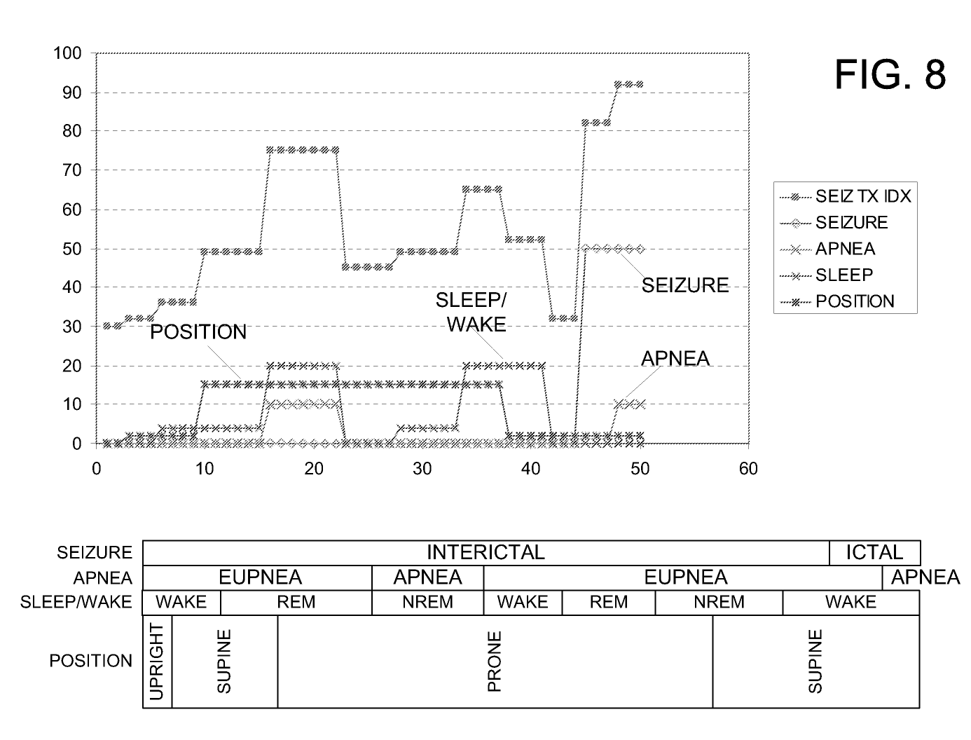

FIG. 8 is a graph illustrating a method to calculate a seizure treatment index using a weighted linear combination of indicator states;

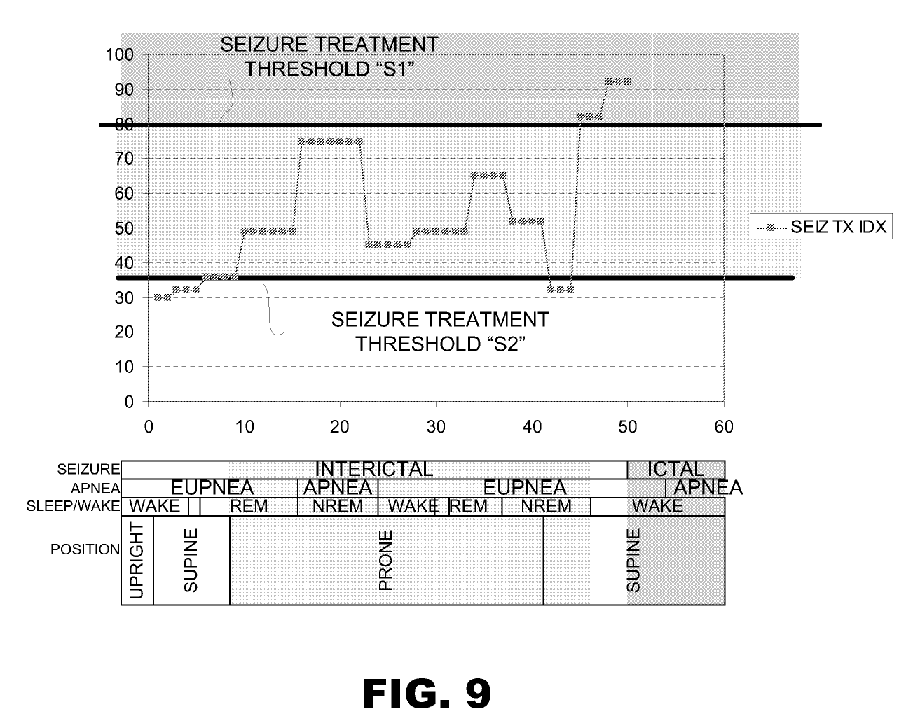

FIG. 9 is a graph illustrating a method to determine appropriate seizure treatments using a thresholding algorithm and the seizure treatment index;

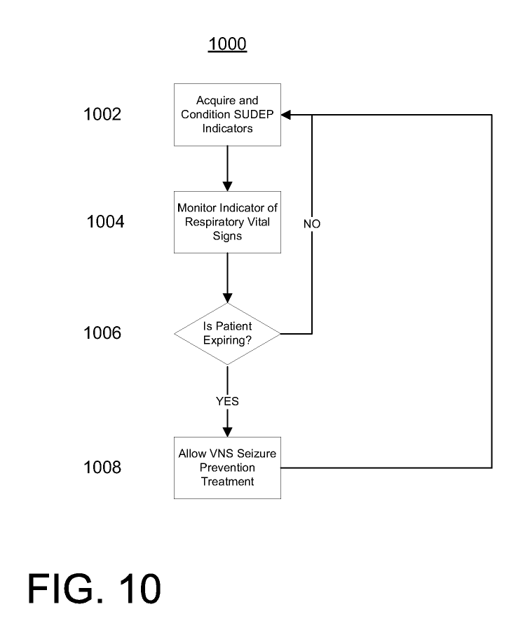

FIG. 10 is a flow chart of a method to circumvent obstructive apnea side effects resulting from vagus nerve stimulation; and

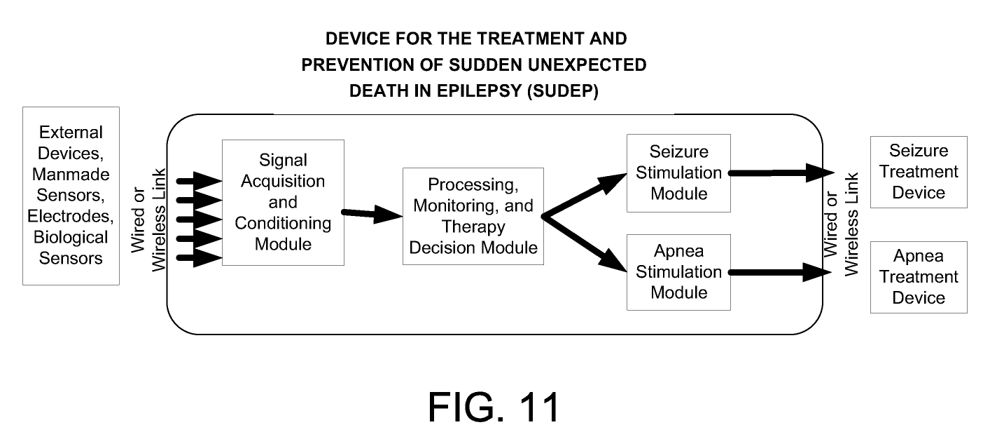

FIG. 11 is a schematic representation of a device to prevent sudden death in epileptic patients.

### DETAILED DESCRIPTION

Generally stated, the non-limitative illustrative embodiment of the present invention provides a method and device for preventing SUDEP.

In the detailed description, unless specified otherwise, reference to the term “apnea” is synonymous with “respiratory failure” and defined to mean an occurrence of obstructive, central, mixed, or complex apnea or hypopnea, occurring during or between seizures, and occurring during the sleeping or waking states.

As used herein, the term “apneic event” encompasses a detected occurrence of apnea or hypopnea in a subject.

As used interchangeably herein, unless specified otherwise, the terms “SUDEP indicator” and “indicator” refer to a physical sign of any one of several physiological conditions that are known risk conditions for SUDEP, which conditions include apnea and seizure activity, but also include but are not limited to a sleeping (versus waking) state, prone (versus supine) position, non-REM (NREM) sleep state, and low blood oxygen level. Other SUDEP indicators may be recognized and used according to the present methods provided that the indicators are capable of being physically monitored.

As used herein, the term “SUDEP-related event” refers to the occurrence of a risk condition for SUDEP or co-occurrence of two or more risk conditions for SUDEP to generate a sufficiently high measure of SUDEP risk such as a SUDEP index of a predetermined value, to warrant a treatment such as apnea treatment or seizure treatment or a combination of apnea and seizure treatment, wherein each treatment may be preventative or therapeutic.

A method and device to detect, identify, and treat obstructive and central apneas based on neural recording and stimulation techniques is described in U.S. patent application Ser. No. 12/273,118 filed Nov. 18, 2008, the disclosure of which is herein incorporated by reference in its entirety. A method and system for monitoring respiratory activity and for treatment of breathing disorders such as apnea is described in International Publication No. WO 2008/046190, the disclosure of which is also herein incorporated by reference in its entirety.

### Methods

Referring to FIG. 1, there is shown a flow chart of a general method 100 for the prevention of SUDEP. The method is based in part on the close relationship between apnea and seizure in leading to SUDEP. The method prevents SUDEP by identifying occurrence of high-risk conditions related to apnea or seizure or both, and depending on the coincidence of certain other risk factors, signaling the need for a SUDEP preventive treatment that may involve preventative or therapeutic treatment for apnea, and preventative or therapeutic treatment for seizure. Method 100 thereby prevents SUDEP by providing normal recovery from either or both such events. Also provided are systems for implementing method 100 as well as other methods described herein.

Referring again to FIG. 1, method 100 involves continuous acquisition 102 of at least two indicators that reflect the likelihood of SUDEP. Acquisition involves obtaining an electrical signal representing each of the indicators. It is contemplated that other SUDEP indicators may be realized and used according to the present methods.

In an exemplary embodiment, at least two indicators (though not limited to two) and their corresponding signals are electronically monitored 104 and a SUDEP index is calculated to determine 106 the likelihood of SUDEP occurrence. A predetermined value of the SUDEP index (a threshold) is selected to correspond to the occurrence of a SUDEP-related event warranting a treatment. For example, the SUDEP index may be based on a scale of 0-100 in arbitrary units, and the SUDEP index threshold selected as a single value in the range between, for example, 40 to 50. Typically the method will be implemented on a computer and the value will be a single floating point value in the range between, for example, 40 to 50. The occurrence of any one individual risk condition may be assigned a single value from 0-100 based on the physician or caregiver's professional judgment and knowledge of the particular subject, or each risk condition may be preprogrammed as a fixed value. Whether the value for each condition is selected by a physician or caregiver or preprogrammed, exemplary values for various conditions on a scale of 0-100 are as follows: apnea 20, seizure 40, NREM sleep 15, REM sleep 8, prone (body) position 4, supine (body) position 2 and other conditions such as eupnea, awake state, upright position and lack of seizure activity being assigned a value of 0. These values are merely exemplary and it will be understood that a value for each condition can vary within a range that is selected to reflect relative contribution of the particular condition to the occurrence of SUDEP according to generally recognized medical principles. For example, it is envisioned that on a scale of 0-100, suitable values for apnea could be from 10 through 40, for seizure from 20 through 50, for NREM sleep from 10 through 30, for REM sleep from 1 though 5, for prone (body) position from 1 through 10, and for supine (body) position from 5 through 15. However, it will be recognized that various scales can be used, and different SUDEP index thresholds selected. For example, in a subject known to have a history of SUDEP-related events given the occurrence of seizure, a relatively low threshold of about 35 may be selected. Alternatively or in addition, the method may be in other ways intentionally biased in view of the particular subject's history, for example by adding in a patient risk factor as described elsewhere herein, or by assigning a relatively high value to the occurrence of a condition which, for the particular subject, is an especially high risk condition.

In an exemplary embodiment, a SUDEP-related event is the co-occurrence of any of at least two risk conditions for SUDEP. Alternatively, the method can also accommodate signals indicating duration and severity of a particular condition, so that a sufficiently severe or prolonged apneic event on its own, or a sufficiently severe or prolonged seizure event on its own can be assigned a relatively higher value on the scale than a less severe or shorter duration such condition. In any case, when the SUDEP index meets the predetermined value, a signal is generated 108 indicating occurrence of a SUDEP-related event. Typically the predetermined (threshold) value for the SUDEP index will be about 40 to about 50 on a scale of 0-100. In any event, the SUDEP event signal can then be used to trigger or deliver 110 a SUDEP treatment.

Referring now to FIG. 2, there is shown a block diagram of an exemplary process reflecting method 100, in which a plurality of SUDEP indicators 202 generate electronic signals that are monitored and processed 204 by a central control unit that is typically a computer. The central control unit may be implanted or external to the body. Exemplary SUDEP indicators are derived from electronic monitoring of sleep/wake (REM versus non-REM or “NREM” sleep), body position, respiratory state (expiration versus inhalation), apneic state (eupnea, hypopnea or apnea), blood oxygenation level, and brain activity for seizure state. For a particular subject having a known risk of SUDEP because of previously experienced SUDEP events or for any other reason, an added bias or weighting factor can be added in to the SUDEP computation as may be determined in an individual basis by the subject's medical caregiver. The monitoring and processing 204 of multiple SUDEP indicators enhances the sensitivity of detection of conditions predisposing to SUDEP and assists the selection of the appropriate treatment according to the existing conditions.

In some embodiments, signals of indicators 202 are derived from the output of external devices such as (e.g.) polysomnography systems, electroencephalography systems, pulse oximetry systems, inductive plesmography systems, respirometry systems, or other systems designed to detect and classify sleep state, seizures, or respiratory activity. In other embodiments, indicators also comprise direct monitoring of physiological or behavioral variables, such as the activity of nerve, muscle, biological or manmade sensors. Indicator signals may comprise the raw signal or may be further conditioned using amplification, filtering, integration, or other signal processing methods. Such indicators, systems, sensors, and conditioning methods are known in the art. For example, EEG signals are obtained to detect seizure activity, EEG signals can also be used to detect sleep state and identify whether the subject is in REM sleep or non-REM sleep. Various detector systems such as accelerometers, gyroscopes, pressure and body heat sensors can be used to detect sleep position (prone, supine or upright) of the subject. Apneic state (eupnea, hypopnea or apnea) of the subject can be determined using for example, external respiratory monitors such as thermistors or piezoelectric transducers, or implanted monitors such as monitors of biological pressure sensors as described in U.S. patent application Ser. No. 12/273,118, filed Nov. 18, 2008. Such monitors are well known and can be readily configured to generate the necessary output. Sensors for any indicator may be implanted or external to the body.

In different embodiments, different subsets of indicators 202 are used. For example, in one embodiment, only two indicators 202 are used and are apnea 224 and seizure 230 indicators. In another embodiment, five or more indicators are used including the apnea and seizure indicators, a sleep state indicator 220, one or more body position indicators 222, and a blood oxygen content indicator 228. In another embodiment, a seizure predictor indicator 232 and a patient risk factor indicator 234 may also be used. It is contemplated that other SUDEP indicators may be also be used. Likewise, different subsets of indicators may be used during different operating conditions. For example in an embodiment employing signals from a fully implantable system, the method may involve monitoring only of implantable sensors configured to generate signals specific to selected indicators. In an embodiment employing signals from a partially implantable system, the method may involve monitoring of fully implantable sensors during the waking hours and monitoring by wireless communication with exteriorized, less portable, for example tabletop sensors only at night, while the patient is in bed. In a battery-powered portable embodiment, the patient, physician or caregiver may select a subset of indicators to be donned or doffed daily and connected to the invention. These might vary between waking hours and during the night. In a tabletop embodiment, the patient may use a completely exteriorized version of the system only at night, while the patient is in bed.

Indicators are processed in an activity monitoring and decision process 204 that performs a multiplex operation between indicator inputs and physician-determined treatment output. The decision process combines indicator information to determine if SUDEP is likely, and if so, selects from one of several possible treatments in the form of stimulation of nerves, muscles, or control of external devices that treat or prevent adverse respiratory and/or epileptic events.

In some embodiments, the selected treatment includes both seizure treatment 208 and apnea treatment 210. Specific treatment parameters with respect to apnea treatment and seizure treatment can be predetermined by a physician or medical caregiver taking into the account the patient's history, condition, and risk factors. In an exemplary embodiment, a physician or medical caregiver preselects specific combinations of apnea and seizure therapies for each possible combination of risk factors, and uses the control unit of a system as described herein to preprogram the selections in to the system. Alternatively, the a system implementing the methods can be preprogrammed with selected treatment for each combination of risk factors according to generally recognized standard medical practice. Upon occurrence of a specific set of risk conditions, the system triggers delivery of, and in some embodiments also delivers the preselected treatment. As described elsewhere herein, the treatment may include preventative or therapeutic apnea treatment, and preventative or therapeutic seizure treatment.

In an exemplary embodiment, treatment may further be predetermined to take into account the specific set of symptoms or conditions present in the subject at any given point in time. For example, the method may provide that lower risk events be treated with less invasive therapies. More specifically, the method may provide that when more than one medically sound treatment option exists, a less invasive treatment option is selected when the subject is asleep to avoid unnecessary stress or waking of the patient that might actually exacerbate sleep deprivation and thus increase the risk of SUDEP. Similarly, the method may also provide that when more than one medically sound treatment option exists for sets of conditions that include higher risk events, a more aggressive treatment option is selected to ensure rapid and effective alleviation of dangerous conditions.

Treatments may be defined as preventive or therapeutic. Preventive treatments are designed to maintain the status of patients that have other indicators of SUDEP risk, but may not be currently experiencing apnea or may not be currently experiencing seizure. Therapeutic treatments are designed to put an end to and/or reverse adverse events such as apnea or seizure in patients that are experiencing these adverse conditions. For example, in a subject experiencing apnea but not seizure, a treatment may include a therapeutic treatment for apnea and a preventative treatment for seizure. In a subject experiencing seizure but not apnea, a treatment may include a therapeutic treatment for seizure and a preventative treatment for apnea. In a subject not experiencing either apnea or seizure but exhibiting other SUDEP risk factors nevertheless sufficient to generate an increased probability of SUDEP, the treatment may include preventative treatment of both seizure and apnea. In a subject experiencing both apnea and seizure, the treatment may include therapeutic treatment of both seizure and apnea. Preventive treatment parameters may be deliberatively below the arousal threshold of the sleeping patient. Therapy treatment parameters may be deliberately above arousal threshold of the sleeping patient. The parameters of the combination of apnea and seizure treatments may be deliberately below arousal threshold of the sleeping patient. The parameters of the combination of apnea and seizure treatments may be deliberately above arousal threshold of the sleeping patient.

The method also provides for flexibility in treatment by providing different subsets of treatments according to different operating conditions. For example, for methods and systems using implantable components, treatments may be delivered through implanted seizure or apnea neurostimulation devices. In methods involving tabletop systems, the treatment may be delivered using external devices, such as a continuous positive airway pressure (CPAP) device or external neuromuscular stimulator devices.

Referring still to FIG. 2, the sleep state indicator 220 may use input from an external device or internal logic to determine the current sleep state comprised of (e.g.) the awake, REM sleep, and NREM sleep states. In other embodiments, NREM sleep may be further subdivided into Stage 1, Stage 2, Stage 3 and Stage 4. Sleep state is an important variable in the likelihood and treatment of apnea, seizures, and SUDEP. SUDEP is most likely during sleep, and seizures are most likely during the NREM sleep stages.

The body position indicator 222 may use input from an external device or internal logic in a control unit to determine the current body position comprised of (e.g.) the upright, prone, and supine postures. In an exemplary embodiment of a system for implementing the methods, body position is indicated using an accelerometer, which also can be used in an implanted device. Body position during sleep is an important variable in the likelihood and treatment of apnea, seizures, and SUDEP. Apnea leading to respiratory arrest during seizure is most likely in the prone position, while more benign sleep apneas occur most commonly in the supine position.

The apnea state indicator 224 may use input from an external device or internal logic to determine the current apneic state, which may be one of, for example, obstructive hypopnea, obstructive apnea, central hypopnea, or central apnea. Apneic state is an important variable in the likelihood and treatment of apnea, seizures, and SUDEP. Apneas occurring during sleep may range from relatively benign sleep apneas to potentially fatal ictal apneas. Sleep deprivation from SRBDs including sleep apnea are thought to increase seizure frequency. Frequent seizures are associated with SUDEP.

The respiratory vital signs indicator 226 may use input from an external device or internal logic to determine the current respiratory state, i.e. whether the subject is in expiratory phase or inspiratory phase, and also to indicate tidal volume. Respiratory monitors for providing such signals are well known and readily available from a number of commercial sources. Respiratory vital signs are important variables in the likelihood and treatment of apnea, seizures, and SUDEP. While both obstructive and central sleep apneas typically occur at the beginning of an inspiratory cycle, there is no known relationship between ictal apnea and respiratory cycle. Thus, respiratory state would alter the treatment appropriate to treat ictal apnea (e.g. trigger expiration or inspiration in response to central apnea depending on whether the lungs are full or empty). Respiratory state is also an important determinant in the modulation of VNS stimulation timing to avoid apneic side effects.

The blood oxygenation indicator 228 may use input from an external device or internal logic to determine the current blood oxygenation state, which may be one of, for example, normal saturation, low saturation, rising saturation or falling saturation. It is thought that low blood oxygenation due to apnea may trigger seizure onset and is thought to be the ultimate cause of SUDEP.

The seizure state indicator 230 may use input from an external device or internal logic to determine the current seizure state, which may be, for example, a seizure or a non-seizure state. In some embodiments, the seizure state indicator 230 may also provide a signal indicating the focus or extent of seizure activity. Seizure state is an important variable in the likelihood and treatment of seizures and SUDEP. Evidence of seizure activity is common in SUDEP, and both obstructive and central apneas have been observed during seizure activity.

The patient risk factor indicator 234 includes a physician-determined risk factor for a given patient, which may be, for example, a high, moderate, or low risk state. High, low and moderate risk states can be indicated by assigning a scaled weight or numerical value to each level of risk. A variety of variables are involved in the likelihood of seizures and SUDEP. For example, SUDEP is more common in younger patients, refractory or non-compliant to anti-epilepsy drugs (AEDs), and those with frequent, recent, or severe seizures. For example, a younger (pediatric) patient with a recent seizure may be assigned a high patient risk factor of 30 on a scale of 100, while an older patient with infrequent and no recent seizures may be assigned a low patient risk factor of 0-5 on a scale of 100. A patient with a moderate risk based on personal medical history may be assigned a moderate patient risk factor of 10-15. However, these values are exemplary and the actual values used by a physician or caregiver to indicate a given level of risk factor for a selected patient are determined according to professional judgment and knowledge of the patient, as well as the particular scale being used.

The seizure prediction indicator 234 may use input from an external device or internal logic to determine real-time seizure likelihood comprised of (e.g.) probable, likely, and unlikely states and associated time horizons. Seizure prediction is an important variable in the likelihood of SUDEP. The seizure prediction factor may be derived for example from a device for patient input that the patient triggers when experiencing auras or other indications of impending seizure.

The seizure and/or apnea indicators may be a manual indicator (not shown) that use external input from the patient or caregiver via a switch device or the like to indicate the occurrence of seizure and/or apnea.

Indicator signals may be used directly or further processed before being logically combined to indicate higher-order conditions, likelihood of SUDEP, and best treatment for existing conditions as defined by the physician programmer. It is to be understood that depending on the application there may be other input indicators and/or indicator states from either external devices or internal logic signal sources.

### Process for Determining Likelihood of SUDEP and Selection of Appropriate Treatment

Referring now to FIG. 3, there is shown in a flow diagram one embodiment of a process 300 for the determination of appropriate treatment to prevent SUDEP in epileptic patients.

Process 300 starts at block 302, where indicators such as those described above are acquired and conditioned, after which, at block 304, the indicators are processed and monitored for indicators of SUDEP.

The process continues to block 306, where the process determines if SUDEP is likely based on the status of available indicators and pre-programmed physician settings, using, for example, a SUDEP index or a lookup table.

If it is determined at block 306 that SUDEP not likely, then the process resumes at block 302.

If it is determined at block 306 that SUDEP is likely, then the process proceeds to block 308, where the process determines if apnea treatment is necessary based on the status of available indicators and pre-programmed physician settings, using, for example, an apnea treatment index or a lookup table.

If it is determined at block 308 that apnea treatment is unnecessary, then the process continues to block 320.

If it is determined at block 308 that apnea treatment is necessary, then the process proceeds to block 310, where it is determined if apnea is present using, for example, an apnea indicator, an apnea treatment index or a lookup table.

If it is determined at block 310 that apnea is present, the process proceeds block 312 in which the desired apnea treatment of a therapeutic nature is determined, using, for example, an apnea treatment index or a lookup table, and then on to block 314, where such treatment is triggered or applied.

If it is determined at block 310 that apnea is not present, the process proceeds block 316 in which the desired apnea treatment of a preventive nature is determined, using, for example, an apnea treatment index or a lookup table, and then on to block 318, where such treatment is triggered or applied.

The process then proceeds to block 320, where the process determines if seizure treatment is necessary based on the status of available indicators and pre-programmed physician settings, using, for example, an seizure treatment index or a lookup table.

If it is determined at block 320 that seizure treatment is unnecessary, then the process resumes at block 302.

If it is determined at block 320 that seizure treatment is necessary, then the process proceeds to block 322, where it is determined if seizure is present using, for example, an seizure indicator, a seizure treatment index or a lookup table.

If it is determined at block 322 that seizure is present, the process proceeds block 324 in which the desired seizure treatment of a therapeutic nature is determined, using, for example, an seizure treatment index or a lookup table, and then on to block 326, where such treatment is triggered or applied.

If it is determined at block 322 that seizure is not present, the process proceeds block 328 in which the desired seizure treatment of a preventive nature is determined, using, for example, a seizure treatment index or a lookup table, and then on to block 330, where such treatment is triggered or applied. The process then resumes at block 302.

It is to be understood that the process can operate on an iterative basis and all computations or decisions made on a periodic basis. In an exemplary embodiment, the process is undertaken continuously in real-time and computations are made on a periodic basis as predetermined by a control program.

### EXAMPLES

In the examples described below, in some embodiments indicator values are programmed to retain their value for a fixed time period after the event that triggered them in order to provide memory of given high-risk events. For example, a seizure indicator might remain high for 5 minutes after the end of an ictal event, to increase the probability that combination with relevant other events during this period will trigger treatment. In another embodiment, the extended values are decremented gradually in, for example, a linear or logarithmic fashion over the memory period to create a decreasing probability of treatment over time from the high-risk event.

It is to be understood that the examples are merely representative and that weighted linear combination and thresholding algorithms as described therein will function identically under more realistic conditions in which the subject's state will not progress systematically from low to high-risk states but rather the subject's changing state will likely produce high SUDEP indexes alternating with low SUDEP indexes over time.

It is also to be understood that the indicators, weights, thresholds, and methods of combination are different in different embodiments. For example, in one embodiment, a selected subset of indicators is multiplied before the linear combination step. In one embodiment, multiple ranges of SUDEP index values are selected to indicate different risk conditions. In another embodiment, multiple discontinuous ranges of SUDEP index values are selected as high risk conditions. In yet another embodiment, SUDEP likelihood is evaluated using a lookup table, without relying on the calculation of a SUDEP index or the thresholding method described below.

### Example 1

### SUDEP Index from Seizure, Apnea, Sleep State and Body Position Indicators

FIG. 4 is a graph illustrating in further detail how a method for preventing SUDEP involves determining the likelihood of SUDEP in epileptic patients from acquired SUDEP indicators and using physician-determined variable weighting. In this example, a SUDEP index as an indicator of SUDEP likelihood is calculated based on a set of four SUDEP indicators comprising: a seizure detector, an apnea detector, a sleep state indicator, and a body position indicator. All possible combinations of the available indicator states are shown in the table at the bottom of FIG. 4. The indicator states in FIGS. 4 (and FIG. 5 below) are shown for clarity with increasing weights from left to right.

A SUDEP index was calculated based on the status of the indicators using the simple weighted linear combination process shown in the upper panel of FIG. 4. Here, the variable states for each indicator have been pre-assigned weighting values indicating their contribution to SUDEP likelihood by a physician programmer. A curve for each indicator shows the assigned value for each state along the y-axis in arbitrary units from 0-100. In this example, the “ICTAL” value of the seizure state indicator is assigned the highest weight, followed by the “APNEA” state of the apnea indicator, the “NREM” and “REM’ states of the sleep/wake indicator, respectively, and finally the “PRONE” and “SUPINE” states of the body position indicator, respectively. The “INTERICTAL”, “EUPNIA”, “WAKE”, and “UPRIGHT” states are all assigned the same value (zero) in this example.

An additional “patient risk” indicator (not shown) was assigned a fixed value based the physician's assessment of the patient's history. In this example, the patient risk factor was set to a fixed value of 20 arbitrary units.

After indicators were acquired and weighted, a SUDEP index was calculated by adding together the weighted values for all indicators. It can be seen that this combination of high-risk variable states (e.g. ictal seizure state, apnea, NREM sleep, and prone position) results in a high SUDEP index. A combination of low-risk variable states (e.g. interictal seizure state, eupnea, waking, and upright position) results in a low SUDEP index, and some combination of high, medium and low risk variable states results in intermediate SUDEP values.

In this example, a SUDEP index having a value starting at 40-50 and higher, up to 100 is within a predetermined range that indicates occurrence of a SUDEP-related event, i.e. a set of conditions that indicates the need for a treatment. It can be seen that the SUDEP index value upon reaching the range of 40-50 indicates a subject that has gone into in an apneic state lasting from about t20 to about t35 during which period other SUDEP risk factors are changing over time. At about t20, the subject begins to experience apnea but has just come out of the higher risk NREM sleep. However, during the apneic period, the subject changes body position and eventually cycles back to NREM sleep, so that by the end of the first apneic period but before the subject begins to experience any seizure activity, the SUDEP index has reached a value of about 58 on a scale of 100, indicating occurrence of a SUDEP-related event even though no seizure activity exists.

### Example 2

### SUDEP Likelihood from SUDEP Index

FIG. 5 is a graph illustrating a method of determining if SUDEP is likely using the SUDEP index and a physician determined threshold. Here, indicator inputs resulting in a SUDEP index with values above the threshold (shaded area top), indicate conditions in which SUDEP is considered likely by the physician. The indicator conditions corresponding to suprathreshold values are shown in the shaded area at the bottom of FIG. 5. During programming of a system for preventing SUDEP, the physician has access to a visual display as shown to assist in selecting appropriate weights or/and thresholds in order to tailor the SUDEP index defining high risk values based on patient history and preferred indicator conditions.

### Example 3

### Apnea Therapy Index

FIG. 6 is a pair of graphs illustrating a method using indicator weighting and linear combination to calculate an apnea therapy index to determine application of apnea therapy. Here again, indicator states are shown in the table in the bottom of the figure, and are selected in this example to represent a more natural sequence of states.

In this example, indicators for the apnea therapy index are the same as those available for the SUDEP index in example above, but different weights were applied as predetermined by a physician programmer. In this example, higher relative weights have been assigned to apnea, the supine body position, and REM sleep state.

FIG. 7 is a pair of graphs illustrating use of the method to determine apnea treatment using physician predetermined thresholds and the apnea therapy index. Here, indicator inputs resulting in an apnea index above the apnea threshold labeled “a1” (dark shaded area top) indicate conditions in which a first apnea treatment “A1” will be applied (dark shaded area bottom). Indicator inputs resulting in an apnea therapy index below the apnea threshold labeled “a1” and above the apnea threshold labeled “a2” (light shaded area top graph) indicate conditions in which a second apnea treatment “A2” will be applied (light shaded area bottom graph).

In one embodiment, apnea threshold “a1” is used to detect conditions in which apnea is present by selection of appropriate weights or/and thresholds by the physician programmer to tailor the apnea treatment index. In this case, apnea treatment “A1” corresponds to therapeutic apnea treatment and apnea treatment “A2” corresponds to preventive apnea treatment. Referring back to FIG. 3, block 308 then comprises a process determining if the apnea treatment index exceeds threshold “a2”, and block 310 comprises a process determining if the apnea treatment index exceeds threshold “a1”.

### Example 4

### Seizure Therapy Index

FIG. 8 is a pair of graphs illustrating an example of a weighted linear combination method to calculate a seizure therapy index used to determine application of seizure therapy. The indicator states shown in the table in the bottom of the figure are identical to those shown for the calculation of the seizure therapy index in FIG. 7.

In this example, indicators for the seizure therapy index were the same as those available for the SUDEP and apnea therapy indexes in the examples above, but different weights were applied as predetermined by the physician programmer. In this example, higher relative weights have been assigned to ictal seizure state, the prone body position, and NREM sleep state.

FIG. 9 is a pair of graphs illustrating use of the method to determine a seizure treatment using physician predetermined thresholds and the seizure therapy index. Here, indicator inputs that result in a seizure therapy index above the seizure threshold labeled “S1” (dark shaded area top graph) indicate conditions in which a first seizure treatment “S1” is indicated (dark shaded area bottom graph). Indicator inputs resulting in a seizure therapy index below seizure threshold labeled “S1” and above the seizure threshold labeled “S2” (light shaded area upper graph) indicate conditions in which a second seizure treatment “S2”will be applied (light shaded area bottom graph).

In one embodiment, seizure threshold “s1” is used to detect conditions in which seizure is present by selection of appropriate weights or thresholds or weights and thresholds by the physician programmer to tailor the seizure treatment index. In this case, seizure treatment “S1” corresponds to therapeutic seizure treatment and seizure treatment “S2” corresponds to preventive seizure treatment. Referring back to FIG. 3, block 320 then comprises a process determining if the seizure treatment index exceeds threshold “s2”, and block 322 comprises a process determining if the seizure treatment index exceeds threshold “s1”.

It is to be understood that any number of thresholds and corresponding treatments may be applied to the SUDEP index, seizure therapy index, and apnea therapy index.

In one embodiment, indicator weights and thresholds are set such that SUDEP index is above threshold during all conditions indicating seizure and/or apnea. In one embodiment, indicator weights and thresholds for the SUDEP, apnea therapy index and seizure therapy index are controlled together such that all conditions resulting in a suprathreshold SUDEP index will also trigger seizure or apnea treatment.

Apnea Treatment

Apnea treatment may be comprised of implanted or external devices and is designed to occur automatically under conditions likely for SUDEP with or without patient or caregiver intervention. Examples include devices capable of electrical stimulation of the nerve or muscle, drug delivery, or atrial overdrive pacing. Nerve stimulation treatments include treatments designed to increase airway patency, cause inspiratory muscle contraction, cause expiratory muscle contraction, or elicit complex activity patterns such as swallow, negative pressure reflex, gag, cough, etc. by activating CNS central pattern generators

The invention allows for different treatments of apnea depending on the physiological state of the patient. This flexibility may be based on the type, timing, duration, or amplitude of apnea and could expand to other physiologically relevant parameters such as sleep position, blood oxygenation, phase of sleep, etc.

Flexible or adaptive apnea treatments are necessary because the physiological response to treatment will vary with physiological condition.

For example, the tone of the airway musculature is known to differ between waking, REM sleep, and NREM sleep. In addition, constriction and desynchronization of the airway musculature is known to occur during seizure in animal models. These factors would influence the response to apnea treatment. Thus, an apnea treatment designed to open the airway without awakening a sleeping patient might be insufficient to open an airway constricted during seizure. Conversely, an apnea treatment sufficient to open the airway during seizure would likely awaken the patient with ‘normal’ sleep apnea in the absence of seizure.

Flexible or adaptive apnea treatments also are necessary because the desired response may be preventive or therapeutic.

Treatment options may include differences in the type, timing, duration, and/or amplitude of apnea treatment. Treatment types and parameters are preselected by a physician based on monitored physiological conditions, patient history, and known relationships between sleep, seizure, and fatal apnea. Such apnea treatment methods are known in the art and described for example in.

Seizure Treatment

Seizure treatment may be comprised of implanted or external devices and is designed to automatically trigger seizure treatment under conditions likely for SUDEP with or without patient or caregiver intervention. Examples include devices capable of drug delivery, electrical stimulation of the vagus nerve, electrical stimulation of the cortex, or electrical stimulation of the deep brain structures.

In addition, the invention allows for different treatments of seizure depending on the physiological state of the patient. This flexibility may be based on the type, timing, duration, or amplitude of seizure and could expand to other physiologically relevant parameters such as sleep position, blood oxygenation, phase of sleep, etc.

Flexible or adaptive seizure treatments are necessary because the physiological response to treatment will vary with physiological condition.

For example, cortical synchronization is thought to promote seizure initiation and is known to differ between waking, REM sleep, and NREM sleep. Moreover, it has been suggested that the short-term drops in cortical oxygenation caused by apnea may trigger seizures. These factors would influence the response to seizure treatment. Thus, a seizure treatment designed to terminate seizure in an awake and normally breathing patient might be insufficient terminate a seizure triggered by apnea in NREM sleep. Given the interaction between apnea and seizure, seizure treatment alone may also be insufficient in this case.

Flexible or adaptive seizure treatments also are necessary because the desired response may be preventive or therapeutic.

Treatment options may include differences in the type, timing, duration, and/or amplitude of seizure treatment. Treatment types and parameters are preselected by a physician based on monitored physiological conditions, patient history, and known relationships between sleep, seizure, and fatal apnea. Such seizure treatment methods are known in the art.

Interventional Treatment

In one embodiment, treatment includes an intervention treatment block that may be comprised of implanted or external devices designed to promote corrective action by the patient or caregiver under conditions likely for SUDEP with or without previous patient or caregiver intervention. In one embodiment, high-risk conditions would initiate audible, visible, or tactile warnings to the patient and/or caregiver. Appropriate corrective action may include waking or repositioning the patient, manually initiating AED or other seizure treatment, applying oxygen treatment, applying CPAP treatment, or preparing to administer resuscitation if necessary. Intervention treatment may be applied alone, or in combination with other therapies as described above.

Flexible or adaptive intervention treatments are necessary because the physiological response to intervention treatment will vary with physiological condition.

Flexible or adaptive intervention treatments also are necessary because the desired response may be preventive or therapeutic.

Treatment options may include differences in the type, timing, duration, and/or amplitude of intervention treatment.

Such intervention treatment methods are known in the art.

Prevention of OSA Side-Effects in VNS Stimulation

It has been shown that in addition to the desirable stimulation of vagus nerve afferent fibers for therapeutic treatment of epilepsy, vagus nerve stimulation also stimulates vagus nerve efferent fibers that innervate muscles of the upper airway. This causes undesirable constriction of airway musculature and decreased airflow, particularly during stimulation, which may block airflow during inspiration. Given the interrelationship between epilepsy and apnea, obstructive apnea is a particularly unfortunate side effect of VNS stimulation for epilepsy.

Referring now to FIG. 10, there is shown in a flow diagram a process 1000 for the control of interictal VNS timing, designed to reduce the occurrence of obstructive sleep apnea as a side effect of VNS stimulation. The steps composing the process are indicated by blocks 1002 to 1008.

The process 1000 starts at block 1002, where indicators such as those described above are acquired and conditioned.

Here, the set of included indicators include at least one respiratory vital signs indicator capable of providing indications of respiratory phase and/or amplitude and/or tidal volume during normal respiration.

The process 1000 continues to block 1004 and block 1006, where the process determines if the patient is in the expiratory phase of respiration based on the status of available indicators and pre-programmed physician settings.

If it is determined at block 1006 that the phase of respiration is not expiratory, then the process resumes at block 1002.

If it is determined at block 1006 that the phase of respiration is expiratory, then the process proceeds to block 1008, where the process communicates to an external VNS stimulation device, and allows VNS stimulation to proceed. The process then resumes at block 1002.

Systems

Also provided are systems for implementing the methods for preventing SUDEP as described herein. Such a system, for example, includes multiple modules operatively coupled to one another, for example through a control module including software for processing the multiple inputs and for generating signals for delivering one or more SUDEP treatments. In an exemplary embodiment, the control module is configured to employ digital logic and programming and acquires indicators using analog-to-digital conversion or digital input/output processes. The system may also comprise wireless communication devices, amplifiers, filters, A/D converters, input/output buffers, processor and data clocks, digital signal processor or other logical devices, memory, communication buses, etc. Upon signal acquisition, indicators are optionally conditioned through amplification, filtering, rectification and bin integration, or other processes known in the art. Conditioned indicators provide input to the control module, which monitors and processes the indicator signals, and generates an output signal(s) that represents at least a therapy decision and may also represent a specific instruction to a treatment component or device to deliver a selected treatment.

The control module is typically a computer programmed to use digital logic according to the methods described herein to determine the current status of the patient state based on the indicator input and to determine the preferred output associated with that patient state. The programming is in one embodiment configured to allow the input of a physician or other medical caregiver to modify treatment parameters depending on specific patient factors such as previous general medical history and specifically with respect to apnea and seizure, age, weight, etc.

An exemplary system includes an apneic event detection module for detecting an apneic event, i.e. an occurrence of hypopnea or apnea. The module is operatively coupled to one or more apnea or hypopnea sensors. It is contemplated that different types of apnea or hypopnea sensors can be used to generate a suitable electronic signal. An exemplary such sensor is an electrode configured to be placed in contact with a nerve of the subject to record electrical signals from the nerve to provide an electroneurogram signal. For example, the nerve is the superior laryngeal nerve, and the electrode is configured to be placed in contact with the internal branch thereof. The electroneurogram signal is then typically conditioned by elements in the control module. The control module is further configured for example using a software program to use the electroneurogram signal to generate an output that reports the occurrence of an apneic event. For example, a control unit programmed to compute an apnea indicator would process the electroneurogram signal, compute an indicator of respiratory activity, and when the respiratory activity meets a predetermined value, the control unit reports an occurrence of an apneic event.

The system also includes at least one other module for processing input from at least one other sensor configured to generate an electronic signal corresponding to at least one other SUDEP indicator. In an exemplary embodiment, the system includes a seizure event detection module operatively coupled to the control module. The seizure event detection module is programmed to detect presence of seizure activity from an EEG signal from the subject, and further programmed to generate an electronic signal representing presence of seizure activity in the subject. The EEG signal may also be conditioned by elements in the control unit that is also programmed to compute an index such as a seizure index or other output that reports the occurrence of seizure activity. For example, when the control unit computes an index that meets a predetermined value, the unit reports an occurrence of a seizure. Alternatively or in addition, the control unit may be configured to process the EEG signal to indicate pre-seizure activity.

In accordance with the methods of the invention, the system may also include one or more additional sensors each configured to obtain a physical signal from the subject that indicates occurrence in the subject of one other SUDEP-related condition or risk factor including sleep state, sleep position, respiratory state (point in respiratory cycle as distinguished from apneic indicator), blood oxygen content, and pre-seizure activity. Sensing devices for such conditions are well-known, including for example EEG devices, pressure and heat sensors, respirometers, etc.

The control unit is typically configured to acquire the electrical signals continuously and in real-time, and to run a process embodying the methods of the present invention continuously and in real-time or at least on a regular periodic basis. In one embodiment, the system is configured to evaluate indicators at a rate equal to, or at some fraction of the signal acquisition rate.

The system is configured to use the sensor inputs to generate an output instructing delivery of an apnea treatment, a seizure treatment, or both. Output may comprise trigger signals or instructional signals to external treatment delivery devices or alternatively in certain instances may comprise signals for direct delivery of therapy from the control device or through sensors. For example, in one embodiment, the control module is further configured to generate stimulation signals in response to the report of an apneic event and/or seizure activity. More specifically, the control module is configured to generate a stimulation signal for stimulating the nerve of the subject when an apneic event is detected. The stimulation signal for the nerve can be selected to have a predetermined frequency and amplitude in accordance with treatment parameters determined by a physician or other medical caregiver. The control module is also configured to generate a second stimulation signal for stimulating the nervous system of the subject when seizure activity is detected. The control unit is typically configured to generate the second stimulation signal with a second amplitude and a second frequency that are different than the amplitude and frequency of the nerve stimulation signal generated in response to the detection of an apneic event. The frequency and amplitude of the second stimulation signal can also be selected to in accordance with treatment parameters determined by a physician or other medical caregiver.

In another embodiment, a system for preventing SUDEP in a subject includes multiple sensors each generating an electrical signal representative of a SUDEP indicator, and a control module operatively coupled to the sensors. The control unit is configured to acquire and process the electrical signals from the sensors to compute the SUDEP index as described herein. The control unit may further be configured to generate an electrical signal representing a SUDEP alert when the SUDEP index meets a predetermined value. For example, the control unit may be programmed to compute the SUDEP index simply by using Boolean logic, i.e. assigning one of two possible values to each SUDEP indicator wherein the assigned value indicates presence or absence of a condition corresponding to the SUDEP indicator; and performing a logical AND operation on the values assigned to the plurality of SUDEP indicators to obtain the SUDEP index. When the SUDEP index reaches a predetermined value, e.g. a value indicating co-occurrence of at least three SUDEP risk conditions, the control module generates a SUDEP alert and may also generate a signal instructing and/or implementing delivery one or more SUDEP treatments to the subject. Typically the control module will be programmed to reiteratively acquire the electrical signals from the sensors periodically re-compute the SUDEP index in real time.

In one embodiment the system will include a SUDEP treatment device operatively coupled to the control unit. For example, the system may include a SUDEP treatment device in the form of an apnea treatment component or a seizure treatment component or both. An apnea treatment component is, for example, selected from several possible devices such as a nerve stimulation device, a muscle stimulation device, an apnea drug delivery device, an atrial overdrive pacing device and a waking alarm. A seizure treatment component is for example a seizure drug delivery device, a nerve stimulation device and a brain stimulation device.

The treatment devices are configured to provide therapeutic or preventative treatment for apnea and therapeutic or preventative treatment for seizure, subject to the output of the control module with respect to occurrence of an apneic event and occurrence of seizure. For example, when the system signals occurrence of apnea in the subject, the control unit is configured to generate a signal instructing delivery of a therapeutic treatment for apnea. Such a signal is, for example, a signal to a nerve stimulation device to begin nerve stimulation. Similarly, when the system signals occurrence of seizure activity in the subject, the control unit is configured to generate a signal instructing delivery of a therapeutic treatment for seizure. Such a signal is, for example, a signal to a brain stimulation device to begin anti-seizure brain stimulation. The system thus can be readily configured to differentially respond to a sufficiently high SUDEP index depending on whether only apnea, only seizure, both apnea and seizure or neither apnea nor seizure are detected in the subject. That is, the control unit is programmed so that: when only apnea but not seizure is detected in conjunction with a sufficiently high SUDEP index, the control unit responds by generating an instruction to deliver a therapeutic treatment for apnea and a preventative treatment for seizure; when only seizure but not apnea is detected in conjunction with a sufficiently high SUDEP index, the control unit responds by generating an instruction to deliver a therapeutic treatment for seizure and a preventative treatment for apnea; when both apnea and seizure are detected in conjunction with a sufficiently high SUDEP index, the control unit responds by generating an instruction to deliver a therapeutic treatment for apnea and a therapeutic treatment for seizure; and when neither apnea nor seizure are detected yet a sufficiently high SUDEP index is computed, the control unit responds by generating an instruction to deliver a preventative treatment for apnea and a preventative treatment for seizure.

In another embodiment, the system is configured to prevent SUDEP in a subject being treated for epilepsy with Vagal Nerve Stimulation (VNS). The system includes at least one sensor configured to indicate the respiratory phase (expiration or inhalation) of the subject. The control unit is configured to generate an electrical signal representing a command to periodically deliver the VNS to the subject in synchrony with an expiratory phase of the subject's respiratory state. The system may further include a VNS device operatively coupled to the control unit.

Referring to FIG. 11, there is shown a block diagram of a system 1100 for the prevention of apnea and/or seizure leading to SUDEP. The system may include the various elements in a variety of form factors. For example, the whole system may be a battery-powered implantable device, or a portable battery-powered device carried externally by the patient in a pocket or backpack, or a tabletop system. Communications among the various elements, for example between the sensor inputs and the control module, may be wireless or hard-wired. Each of the multiple sensors may implanted or external sensors, independent of whether the other sensors are implanted or external. For example, the system may include implanted wireless sensors for apnea, and external hard-wired sensors for seizure.

Although the present invention has been described by way of illustrative embodiments and examples thereof, it should be noted that it will be apparent to persons skilled in the art that modifications may be applied to the present particular embodiment without departing from the scope of the present invention.

What is claimed is:

1. A method to prevent SUDEP in a subject in need thereof comprising:
acquiring from the subject an electrical signal representing each of at least two SUDEP indicators;
using the electrical signals to compute a SUDEP index; and
when the SUDEP index meets a predetermined value, generating an electrical signal representing occurrence of a SUDEP-related event.

2. A method according to claim 1 wherein acquiring the electrical signals representing each of the at least two SUDEP indicators occurs continuously in real-time and the SUDEP index is computed periodically.

3. A method according to claim 1 wherein each of the at least two SUDEP indicators indicates a condition selected from the group consisting of: sleep state, sleep position, apneic (respiratory) state, sleep state, body position, apneic state, respiratory state, blood oxygen content, seizure activity and pre-seizure activity.

4. A method according to claim 1 further comprising generating a signal instructing delivery of a SUDEP treatment to the subject.

5. A method according to claim 4 wherein the SUDEP treatment is selected from the group consisting of apnea treatment, seizure treatment, or a combination thereof.

6. A method according to claim 5 wherein apnea treatment comprises preventative treatment for apnea or therapeutic treatment for apnea.

7. A method according to claim 5 wherein seizure treatment comprises preventative treatment for seizure or therapeutic treatment for seizure.

8. A method according to claim 4 further comprising delivering the SUDEP treatment to the subject.

9. A method according to claim 5 wherein delivering a SUDEP treatment to the subject comprises generating an electrical signal representing a command for automated delivery of the SUDEP treatment to the subject.

10. A method according to claim 1 further comprising determining when one of the at least two SUDEP indicators is an apneic event indicator and generating an electrical signal representing presence or absence of apnea in the subject.

11. A method according to claim 10 wherein when presence of apnea in the subject is signaled, the SUDEP treatment includes therapeutic treatment for apnea.

12. A method according to claim 1 further comprising determining when one of the at least two SUDEP indicators is an apneic event indicator and generating an electrical signal representing presence or absence of hypopnea in the subject.

13. A method according to claim 12 wherein when presence of hypopnea in the subject is signaled, the SUDEP treatment includes preventive treatment for apnea.

14. A method according to claim 1 further comprising determining when one of the at least two indicators is a seizure indicator and generating a signal representing presence or absence of seizure in the subject.

15. A method according to claim 12 wherein when presence of seizure in the subject is signaled, the SUDEP treatment includes therapeutic treatment for seizure.

16. A method according to claim 1 further comprising conditioning the electrical signals before using the electrical signals to compute the SUDEP index.

17. A method to prevent SUDEP in a subject in need thereof, the method comprising:
acquiring from the subject an electrical signal representing each of at least two SUDEP indicators;
using the electrical signals to compute a SUDEP index;
determining when one of the at least two SUDEP indicators is an apnea indicator and generating an electrical signal representing presence or absence of apnea in the subject;
determining when one of the at least two SUDEP indicators is a seizure indicator and generating an electrical signal representing presence or absence of seizure in the subject;
when the SUDEP index meets a predetermined value, generating a signal representing occurrence of a SUDEP-related event; and
when apnea is present and seizure is absent, generating an electrical signal instructing delivery of a SUDEP treatment comprising therapeutic treatment for apnea and preventative treatment for seizure.

18. A method according to claim 17 further comprising delivering to the subject the SUDEP treatment comprising therapeutic treatment for apnea and preventative treatment for seizure.

19. A method according to claim 18 wherein at least one of the at least two SUDEP indicators is a sleep state indicator, and wherein the SUDEP treatment is selected to maintain the sleep state of the subject.

20. A method to prevent SUDEP in a subject in need thereof, the method comprising:
acquiring from the subject an electrical signal representing each of at least two SUDEP indicators;
using the electrical signals to compute a SUDEP index;
determining when one of the at least two SUDEP indicators is a seizure indicator and generating an electrical signal representing presence or absence of seizure in the subject;
determining when one of the at least two SUDEP indicators is an apnea indicator and generating an electrical signal representing presence or absence of apnea in the subject;
when the SUDEP index meets a predetermined value, generating a signal representing occurrence of a SUDEP-related event;
when apnea is absent and seizure is present, generating an electrical signal instructing delivery of a SUDEP treatment comprising preventative treatment for apnea and therapeutic treatment for seizure.

21. A method according to claim 20 further comprising delivering to the subject the SUDEP treatment comprising preventative treatment for apnea and therapeutic treatment for seizure.

22. A method according to claim 21 wherein at least one of the at least two SUDEP indicators is a sleep state indicator, and wherein the SUDEP treatment is selected to maintain the sleep state of the subject.

23. A method to prevent SUDEP in a subject in need thereof, the method comprising:
acquiring from the subject an electrical signal representing each of at least two SUDEP indicators;
using the electrical signals to compute a SUDEP index;
determining when one of the at least two SUDEP indicators is a seizure indicator and generating an electrical signal representing presence or absence of seizure in the subject;
determining that one of the at least two SUDEP indicators is an apnea indicator and generating an electrical signal representing presence or absence of apnea in the subject;
when the SUDEP index meets a predetermined value, generating a signal representing occurrence of a SUDEP-related event;
when apnea is present and seizure is present, generating an electrical signal instructing delivery of a SUDEP treatment comprising therapeutic treatment for apnea and therapeutic treatment for seizure.

24. A method according to claim 23 further comprising delivering to the subject the SUDEP treatment comprising therapeutic treatment for apnea and therapeutic treatment for seizure.

25. A method according to claim 24 wherein at least one of the at least two SUDEP indicators is a sleep state indicator, and wherein the SUDEP treatment is selected to maintain the sleep state of the subject.

26. A method to prevent respiratory failure in a subject suffering from a seizure, the method comprising:
acquiring from the subject an electrical signal representing each of at least two SUDEP indicators wherein at least one SUDEP indicator is a seizure indicator;
using the electrical signals to compute a SUDEP index; and
when the SUDEP index meets a predetermined value, generating an electrical signal representing occurrence of a SUDEP-related event.

27. A method according to claim 26 wherein at least one of the at least two SUDEP indicators indicate a condition selected from the group consisting of:
sleep state, sleep position, apneic state, and blood oxygen content.

28. A method according to claim 26 further comprising generating a signal instructing delivery of a SUDEP treatment to the subject.

29. A method according to claim 28 further comprising delivering a SUDEP treatment to the subject.

30. A method according to claim 29 wherein delivering a SUDEP treatment to the subject comprises generating an electrical signal representing a command for automated delivery of the SUDEP treatment to the subject.

31. A method according to claim 29wherein the SUDEP treatment is selected from the group consisting of: waking the patient, apnea treatment, seizure treatment, and a combination thereof.

32. A method according to claim 31 wherein when apnea is present the SUDEP treatment includes therapeutic treatment for apnea, and when seizure is absent, the SUDEP treatment includes preventive treatment for apnea.

33. A method according to claim 31 wherein when seizure is present the SUDEP treatment includes therapeutic treatment for seizure, and when seizure is absent, the SUDEP treatment includes preventive treatment for seizure.

34. A method to prevent SUDEP in a subject in need thereof, the method comprising:
wherein the subject is treated with vagal nerve stimulation (VNS), preventing obstructive apnea in the subject by acquiring from the subject an electrical signal representing each of at least two SUDEP indicators wherein at least one of the SUDEP indicators indicates respiratory state;
using the electrical signals to compute a SUDEP index;
when the SUDEP index meets a predetermined value, generating an electrical signal representing a command to periodically deliver the VNS to the subject in synchrony with the expiration of the subject.

35. A system for preventing SUDEP in a subject, comprising:
an apneic event detection module configured to detect presence of an apneic event from an electroneurogram signal from a nerve of the subject by computing an index of respiratory activity, and to generate an electronic signal representing presence of an apneic event when the index of respiratory activity meets a predetermined value;
a seizure event detection module operatively coupled to the apneic event detection module, the seizure event detection module configured to detect presence of seizure activity from an EEG signal from the subject, and to generate an electronic signal representing presence of seizure activity in the subject;
a control module operatively coupled to the apneic event detection module and the seizure event detection module, the control module configured to generate a first stimulation signal and a second stimulation signal when an apneic event and a seizure event are detected, the first stimulation signal having a first amplitude and a first frequency for stimulating the nerve of the subject, the second stimulation signal having a second amplitude and a second frequency for stimulating the central nervous system of the subject, wherein the second amplitude is different than the first amplitude and the second frequency is different than the first frequency.

36. A system for preventing SUDEP in a subject, comprising:
a plurality of sensors, each sensor configured to obtain a physical signal from the subject, wherein the physical signal from each sensor generates an electrical signal representative of a SUDEP indicator; and
a control unit operatively coupled to the plurality of sensors, the control unit configured to acquire the electrical signals from the sensors and use the electrical signals from the sensors to compute a SUDEP index, and to generate an electrical signal representing occurrence of a SUDEP-related event when the SUDEP index meets a predetermined value.

37. A system according to claim 36 wherein the SUDEP index is computed by assigning one of a plurality of predetermined values to each SUDEP indicator wherein the assigned value indicates presence or absence of a condition corresponding to the SUDEP indicator; and performing a logical AND operation on the values assigned to the plurality of SUDEP indicators to obtain the SUDEP index.

38. A system according to claim 36 wherein the control unit is further configured to reiteratively acquire the electrical signals representing each of the at least two SUDEP indicators in real-time and to periodically compute the SUDEP index.

39. A system according to claim 36 wherein each of the plurality of sensors is configured to obtain a physical signal from the subject that indicates a condition selected from the group consisting of: sleep state, sleep position, apneic event, respiratory state, blood oxygen content, seizure activity and pre-seizure activity.

40. A system according to claim 36 wherein the control unit is further configured to generate a signal instructing delivery of a SUDEP treatment to the subject.

41. A system according to claim 40 further comprising a SUDEP treatment device operatively coupled to the control unit.

42. A system according to claim 41 wherein the SUDEP treatment device comprises an apnea treatment component and a seizure treatment component.

43. A system according to claim 42 wherein the apnea treatment component comprises at least one of a nerve stimulation device, a muscle stimulation device, an apnea drug delivery device, an atrial overdrive pacing device and a waking alarm.

44. A system according to claim 44 wherein the seizure treatment device comprises at least one of a seizure drug delivery device, a nerve stimulation device and a brain stimulation device.

45. A system according to claim 30 wherein at least one of the plurality of sensors is an apnea sensor and the system is further configured to generate an electrical signal representing presence or absence of apnea in the subject.

46. A system according to claim 43 wherein when presence of apnea in the subject is signaled, the SUDEP treatment includes therapeutic treatment for apnea.

47. A system according to claim 36 wherein at least one of the plurality of sensors is a seizure sensor and the system is further configured to generate an electrical signal representing presence or absence of seizure in the subject.

48. A system according to claim 47 wherein when presence of seizure in the subject is signaled, the SUDEP treatment includes therapeutic treatment for seizure.

49. A system for preventing respiratory failure during seizure in a subject at risk of seizure, the system comprising:
a plurality of sensors, each sensor configured to obtain a physical signal from the subject, wherein the physical signal from each sensor generates an electrical signal representative of a SUDEP indicator, wherein at least one sensor is a seizure sensor configured to generate an electrical signal indicating presence or absence of a seizure in the subject; and
a control unit operatively coupled to the plurality of sensors, the control unit configured to acquire the electrical signals from the sensors and use the electrical signals from the sensors to compute a SUDEP index, and further configured to generate an electrical signal representing occurrence of a SUDEP-related event when the SUDEP index meets a predetermined value and the seizure sensor indicates presence of a seizure.

50. A system for preventing SUDEP in a subject being treated with Vagal Nerve Stimulation (VNS), the system comprising: a plurality of sensors, each sensor configured to obtain a physical signal from the subject, wherein the physical signal from each sensor generates an electrical signal representative of a SUDEP indicator, wherein at least one sensor is a sensor configured to indicate respiratory state of the subject; and
a control unit operatively coupled to the plurality of sensors, the control unit configured to acquire the electrical signals from the sensors and use the electrical signals from the sensors to compute a SUDEP index, and further configured to generate an electrical signal representing a command to periodically deliver the VNS to the subject in synchrony with an expiratory phase of the subject's respiratory state.

51. A system according to claim 50 further comprising a VNS device operatively coupled to the control unit.
